(19) United States
(12) Patent Application Publication
(10) Pub. No.: US 2015/0342462 A1
(43) Pub. Date: Dec. 3, 2015

(54) REGISTRATION METHOD OF TISSUE POSITION AND APPARATUS USING THE SAME

(71) Applicant: CUREXO, Inc, Seoul (KR)

(72) Inventors: Young-bae Park, Cheongju-si (KR); Chang-hun Song, Goyang-si (KR); Jae-jun Lee, Suwon-si (KR)

(21) Appl. No.: 14/723,656

(22) Filed: May 28, 2015

(30) Foreign Application Priority Data
May 30, 2014 (KR) 10-2014-0065694

(51) Int. Cl. A61B 5/00; A61B 6/03; A61B 5/107

(52) U.S. Cl. CPC A61B 5/0071; A61B 5/0075; A61B 5/7246; A61B 5/4848; A61B 5/4504; A61B 5/0077; A61B 5/1079; A61B 6/032; A61B 2576/00

(57) ABSTRACT

Disclosed is a registration method of tissue position and an apparatus using the method. The apparatus according to the present invention comprises an image storage unit storing an examined image associated with a target bone of surgery, a first position measuring unit measuring position values of multiple points on a surface of an exposed bone, a second position measuring unit acquiring an image of the exposed bone, and a registration control unit for performing a first registration between the positions obtained from the examined image associated with the target bone of surgery and the positions measured by the first position measuring unit, and for performing a second registration between the positions of the image acquired as a result of the first registration and the positions of the image acquired by the second position measuring unit.

### BACKGROUND OF THE INVENTION

1. Field of the Invention

The present invention relates to registration of tissue position. More specifically, the present invention relates to a method for registering positions of tissues in robotic surgery and an apparatus using the method.

2. Description of the Related Art

The deepening of a low birthrate and an aging phenomenon is acting as a catalyst in developing robotic industry. As the need for smart robots working instead of people increases, the worldwide robot market is rapidly expanding. The robots can be utilized in various fields, including operations in biologically dangerous regions like the scene of a fire, the reconnaissance in battlefield, and the lengthy surgery.

Among those robots, medical robots have been being developed focused most on user convenience. The main principles in developing medical robots are to provide convenience in using to doctors, to provide no inconvenience to patients, to minimize invasions of patients, to minimize pains of patients, etc. The medical robot technology is a technic field combining BT(Bio-Tech), NT(Nano-Tech), RT(Robot-Tech), and MT(Medical-Tech).

The orthopedic surgery using a robot enables precise bone cutting. For this, there is a need to figure out positions of bones prior to surgery. It also needs to be figured out whether the positions of bones have been changed during surgery, and how much the bones have been moved, if they have been changed.

During surgery, as methods for figuring out the positions of bones, there are method using anatomical shapes, method using the center of joints, method that matches a shape of bone that is obtained from medical images with a shape of bone exposed in surgery, etc.

In this regard, in orthopedic surgery using computer, various sensor systems are used in order to figure out or track the positions of bones. The sensor systems for such purposes have been being used in accordance with their uses, based on properties, such as the degree of precision, the number of markers that can track concurrently, the required space for use.

In some orthopedic surgery, for example, in orthopedic surgery using robots, positions of bones are figured out using medical images and the robots cut bones. When figuring out the positions of bones using medical images, there is a need to measure multiple points on a surface of bone precisely. However, during cutting operations, there is a case to check only whether the bone has been moved.

In this case, if more than two measuring systems are used simultaneously, it would be possible to measure optimally in accordance with each step. However, there is a need to match two measuring systems based on a bone coordinate system.

For this, the coordinate systems of two measuring systems are checkable by installing extra markers on the bone (in general, using a method to fix a bone with screws inserted to the bone). However, it causes damage to the bone.

### SUMMARY OF THE INVENTION

An object of the present invention, which is to overcome aforementioned problems, is to provide a method for registering positions of tissues.

Another object of the present invention is to provide a registration apparatus using the method for registering positions of tissues.

In accordance with one aspect of the present invention, there is provided an apparatus for registering positions of tissues including an image storage unit storing an examined image associated with a target bone of surgery, a first position measuring unit measuring position values of multiple points on a surface of an exposed bone, a second position measuring unit acquiring an image of the exposed bone, and a registration control unit for performing a first registration between the positions obtained from the examined image associated with the target bone of surgery and the positions measured by the first position measuring unit, and for performing a second registration between the positions of the image acquired as a result of the first registration and the positions of the image acquired by the second position measuring unit. In this case, the multiple points could be dyed with a dye to be distinguished from other points on the bone.

The dye could include at least one of a fluorescent material or a food dye, and the fluorescent material could include a fluorescent material emitting infrared rays.

The registration control unit could check the multiple points in real time by the second registration, and could find out whether the bone has been moved during surgery by comparing the image acquired as the result of the first registration with the image acquired by the second position measuring unit.

The first position measuring unit could include a digitizer that has a tip contacted with the multiple points.

The second position measuring unit could include an image sensor having an infrared filter and could include an infrared sensing camera.

In accordance with another aspect of the present invention, there is provided a method for registering positions of tissues including measuring positions of multiple points on a surface of an exposed bone by a first position measuring unit, performing a first registration between positions obtained from an examined image associated with a target bone of surgery and the positions measured by the first position measuring unit, acquiring an image of the exposed bone by a second position measuring unit, and performing a second registration between the positions of an image acquired as a result of the first registration and the positions of the image acquired by the second position measuring unit.

The method for registering positions of tissues could further include a step of checking the multiple points in real time by the second registration.

The method for registering positions of tissues could further include a step of finding out whether the bone has been moved during surgery by comparing the image acquired as the result of the first registration with the image acquired by the second position measuring unit.

According to the present invention, it is possible to establish a reference point for position registration without damaging to a bone in robotic surgery.

In addition, when comparing with conventional physical methods, the time for establishing the reference point of registration is reduced. Thus, it can provide more efficient position registration.

### BRIEF DESCRIPTION OF THE DRAWINGS

FIG. 1 is a schematic diagram of a registration apparatus of tissue position according to the present invention.

FIG. 2 is a conceptual diagram of registration according to the present invention.

FIG. 3A, FIG. 3B, and FIG. 3C notionally illustrate scenes of surgery room performing registration of tissue position according to the present invention.

FIG. 4 is a flowchart that depicts the registration method of tissue position according to the present invention.

### DETAILED DESCRIPTION OF THE INVENTION

Hereinafter, exemplary embodiments of the present invention will be described in detail. However, the present invention is not limited to the exemplary embodiments disclosed below, but can be implemented in various forms. The following exemplary embodiments are described in order to enable those of ordinary skill in the art to embody and practice the invention.

It will be understood that, although the terms first, second, etc. may be used herein to describe various elements, these elements should not be limited by these terms. These terms are only used to distinguish one element from another. For example, a first element could be termed as a second element, and similarly, a second element could be termed as a first element, without departing from the scope of the present invention. The term and/or used herein includes any or all combinations of one or more of the associated listed items.

It will be understood that when an element is referred to as being connected or coupled to another element, it can be directly connected or coupled to the other element or intervening elements may be present.

The terminology used herein is for the purpose of describing particular embodiments only and is not intended to be limiting of the invention. As used herein, the singular forms a, an and the are intended to include the plural forms as well, unless the context clearly indicates otherwise. It will be further understood that the terms comprises, comprising, includes and/or including, when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meanings as commonly understood by one of ordinary skill in the art to which this invention belongs. It will be further understood that terms, such as those defined in commonly uses dictionaries, should be interpreted as having a meaning that is consistent with their meaning in the context of the relevant art and will not be interpreted in an idealized or overly formal sense unless expressly so defined here.

The human body comprises bones, skins, muscles, etc. In the specification, the term “tissues” means a part of tissues of body, and the term “soft tissues” means tissues such as skins and muscles, etc. except bones in the body tissues. The term “images” used herein includes static images and moving images.

Hereinafter, preferred embodiments of the present invention will be described in detail with the accompanying drawings. In the following description, the same reference numerals denote the same elements to facilitate the overall understanding, and repeated description thereof will be omitted.

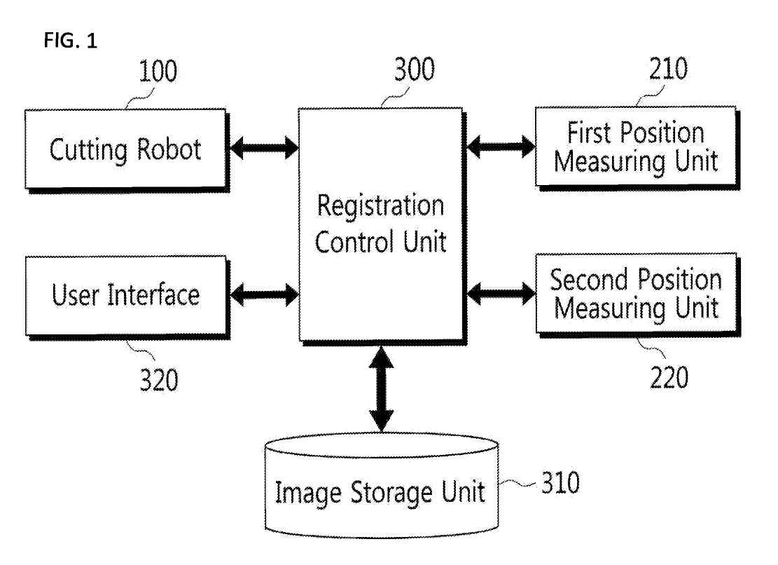

FIG. 1 is a schematic diagram of a registration apparatus of tissue position according to the present invention.

Hereafter, the elements according to the present invention, which will be described by referring to FIG. 1, are those elements defined by functional classification not by physical classification. The elements according to the present invention could be defined by functions performed by each of the elements. Each of the elements could be implemented as hardware and/or program codes performing each function and processing units. They also could be implemented so that the functions of two or more elements are included in one element. Therefore, it needs to be noted that the names of elements, given in following embodiments, are not for distinguishing elements physically, for representing main function performed by each element. Furthermore, it needs to be noted that the spirit of the present invention is not be limited by the names of elements.

As illustrated in FIG. 1, the registration apparatus of tissue position according to the present invention comprises a cutting robot 100, one of the orthopedic surgery robots, to which surgery equipment for cutting bones is attached, an image storage unit 310 storing an examined image associated with a target bone of surgery, a first position measuring unit 210 measuring position values of multiple points on a surface of an exposed bone, a second position measuring unit 220 acquiring an image of the exposed bone, and a registration control unit 300 for performing a first registration between the positions obtained from the examined image associated with the target bone of surgery and the positions measured by the first position measuring unit and for performing a second registration between the positions of the image acquired as a result of the first registration and the positions of the image acquired by the second position measuring unit.

The registration control unit 300 figures out the multiple positions in real time by the second registration, and finds out whether the bone has been moved during surgery by comparing the image acquired as the result of the first registration with the image acquired by the second position measuring unit 220.

The first position measuring unit 210 could be a digitizer that has a tip contacted with the multiple points, and the second position measuring unit 220 could be an image system that includes an image sensor having an infrared filter.

In addition, the registration apparatus of tissue position according to the present invention further comprises a user interface 320 for providing images of surgery region acquired in surgery room to user, and for enabling users to enter commands regarding robotic surgery.

Specifically, the cutting robot 100 cuts the bone according to the plan, which has been established based on the examined three-dimensional images of the target bone of surgery.

The image storage unit 310 stores three-dimensional shape data of bones obtained by computerized tomography equipment, etc.

The first and second position measuring unit 210, 220 measure the position of the bone exposed by incising skins and skin tissues in real surgery. The one example of the first position measuring unit 210 is a digitizer. The second position measuring unit 220 could be an infrared camera system.

Like this, according to the present invention, more than one position measuring unit is used. Although the schematic diagram of FIG. 1 shows a structure that two position measuring units are applied, the position measuring unit could be more than three.

In other words, as the position measuring unit, a digitizer formed with multi joint arms, a camera system using an infrared marker, a camera system using image processing, a sensor system using variations of magnetic fields, etc. could be applied. Each system has individual advantages and disadvantages, such as the level of precision, the number of markers that can track concurrently, the required space for use, etc. Thus, the each system is used to suit own purposes.

The method using more than one image system or one measuring system is called as multi-modal method. In the multi-modal method, it is important to confirm the relationship between modalities. According to the present invention, it uses dyes to determine a reference point defining the relationship of modalities.

Regarding the concept of multi-modality, suppose that information for tracking positions of bones is obtained from two modalities, image analysis and a digitizer. According to the image analysis, although a real-time continual tracking is possible and general movements of positions are obtainable, the absolute accuracy of positions is reduced. On the other hand, according to the digitizer, the absolute accuracy of positions is great. However, it causes inconvenience to the real-time tracking during surgery because the tip of digitizer has to be attached to bones.

In such case, the advantages of each modality can be applied all at once by combining two modalities. The combination of two modalities can be obtained by recognizing the positions that have been measured by the digitizer using images. In this case, the particular position in one modality should be known exactly to the other modality. For this, according to the present invention, using a marker pen, mark a reference point on the periosteum of exposed bone, and perform position registration based on the reference point.

The registration control unit 300 matches three-dimensional shape images of the bone, which is obtained prior to surgery by computerized tomography equipment, etc., with three-dimensional position data obtained in surgery room. Herein, the step of matching the three-dimensional shape images of the bone, which is obtained prior to surgery by computerized tomography equipment, etc., with the three-dimensional position data obtained in surgery room is referred to as registration.

In robotic surgery, the position registration is a step to calculate preferred surgery positions based on the anatomical positions of bones measured by an anatomical position finder and a surgery robot. Although, there are various methods for registration, the most representative registration method will be explained hereafter.

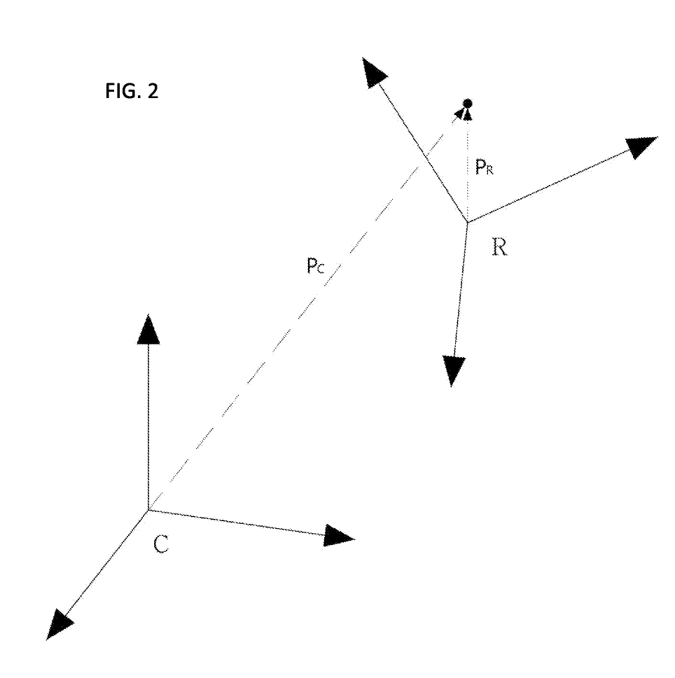

FIG. 2 is a conceptual diagram of registration according to the present invention.

In FIG. 2, C denotes the coordinate system of CT having pre-obtained images of bone by using CT, and R denotes the coordinate system of robot in real space, surgery room. The goal of the registration is to calculate the transformation matrix between the two coordinate systems. When P_{c} denotes one point on the coordinate system C, P_{c}. can be represented as a point on the coordinate system R as the following equation.

P_{R}=T_{C}^{R}·P_{C} <Equation 1>

Herein, T_{C}^{R} represents a transformation matrix that defines a transformation from the coordinate system C into the coordinate system R. By calculating the transformation matrix between the coordinate system C and the coordinate system R, and by applying the calculated transformation matrix to the CT coordinate system, a processing path of robot can be applied appropriately according to the real position of the bone.

As the registration methods for calculating the transformation matrix, there are pin-basis registration, image-basis registration, etc.

According to the pin-basis registration method, prior to surgery, multiple pins are inserted into the bone. Then, CT images are taken, with the pins inserted from a lesion above a bone of a patient. After that, the processing path of robot is determined based on the CT images. In this case, a reference coordinate system of the processing path of robot is established by the pins on the CT images.

As completed the set-up of the processing path of robot, the registration is performed by matching the real pins inserted into the surgical region with the pins on the CT images that are basis of the processing path of robot. Such pin-basis registration method causes pain and discomfort of patients due to multiple pins inserted into a lesion from start to the end of the surgery.

According to the image-basis registration method, meanwhile, the processing path of robot is determined based on CT images of a thighbone of a patient that are obtained prior to surgery. In the early days, the registration was made by matching three-dimensional images obtained from CT images with two-dimensional X-ray images of bones of patients obtained in real surgery. Such method causes many errors in the process of distinguishing tissues like bone tissues, ligaments, etc. and in the process of detecting edges.

To reduce such errors, recently, the registration method that matches a particular point of a pre-surgery CT image with a particular point measured during surgery by a digitizer has been being used. According to the registration method using the digitizer needs to press surface of a thighbone with a tip of measuring pin with a steady pressure in order to measure the particular point of bone tissues with the measuring pin of digitizer in surgery. When pressing the surface of a thighbone, if pressing force is too small, it causes an error in measuring the particular point, and if pressing force is too big, it causes cracks in surface of the bone. Furthermore, it causes discomfort due to many measuring points for reducing the errors, and it causes difficulty for the surgeon to correspond a measuring pin exactly with a measured point guided by a monitor attached to surgery equipment.

The registration methods described above are based on one position measuring unit. As one preferred example of the present invention, in case that more than two position measuring units are applied, more complicated procedures of position registration are needed. Because the registration should be performed by considering more than two position measuring systems.

For this, the registration control unit 300 performs a first registration between the positions in the image acquired in the CT coordinate system and the positions measured by the first position measuring unit 210. Then, the registration control unit 300 performs a second registration between the positions of the image acquired as a result of the first registration and the positions of the image acquired by the second position measuring unit 220. In addition, the registration control unit 300 finds out whether the bone has been moved in surgery by comparing the image acquired as the result of the first registration with the image acquired by the second position measuring unit 220.

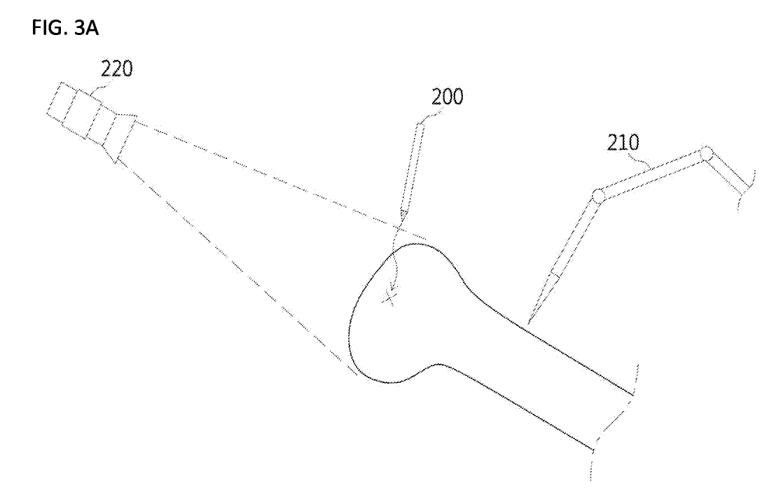

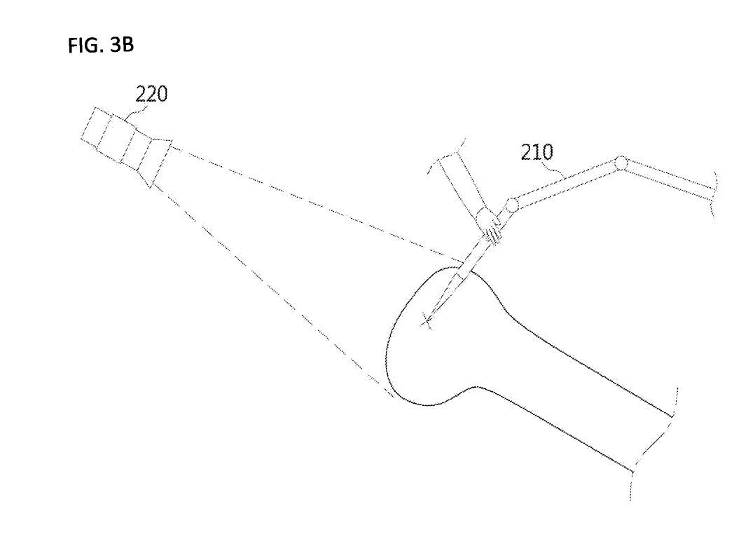

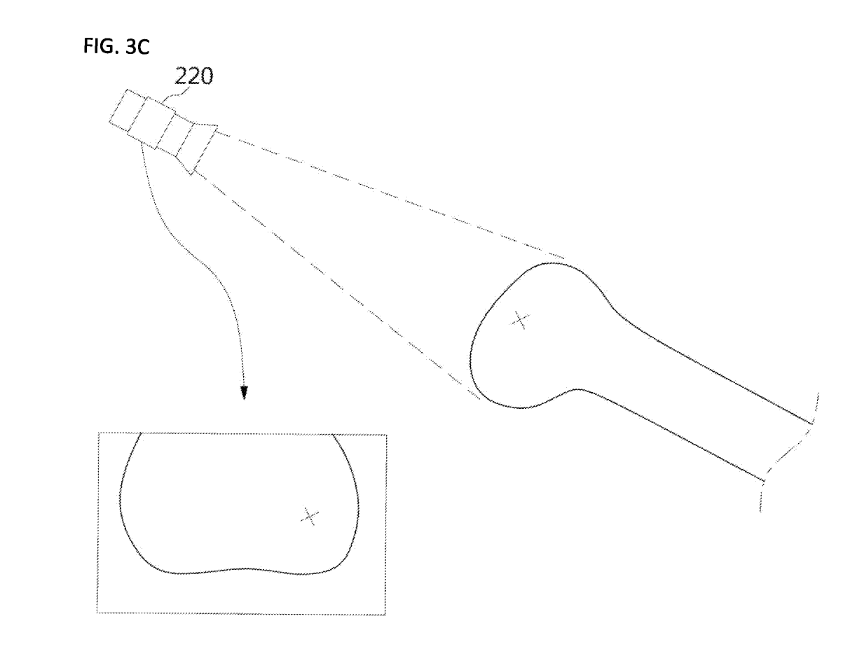

FIG. 3A, FIG. 3B, and FIG. 3C notionally illustrate scenes of surgery room performing registration of tissue position according to the present invention.

The registration system of tissue position according to the present invention comprises a dyeing device dyeing tissues including periosteum of bone surface without harm to human body (a marking pen etc.), and a sensor that enables entering three-dimensional positions (a digitizer, an infrared camera etc.), a camera for obtaining images of surgical region, and a calculating device for image processing.

FIG. 3A shows a scene that marks a distinguishable mark on multiple points of the surface of exposed bone with a marking pen. According to the position registration system, when the bone is exposed in surgery room, it figures out the position of bone. After that, using the marking pen 200, it marks reference points on more than one point of bone, and records three-dimensional positions of the marked points.

FIG. 3B shows a scene that measures the marked points using the digitizer 210. As described above, the digitizer 210 has a role as the first position measuring unit.

FIG. 3C shows a scene that takes an image of bone in real time with the camera 220 and finds out three-dimensional positions of the points marked with the pen in real time using image processing. With these procedures, the present invention can also measure whether the bone has been moved with respect to the camera during surgery.

The registration apparatus of tissue position according to the present invention can figure out where the points marked with respect to the position of bone and the bone are. The reference points for registering positions of tissues can be marked everywhere on the bones. In addition, the reference points can be determined as positions that the surgeon can find accurately due to anatomical unique structures or that can be easily seen during surgery. By the camera system according to the present invention, the marked points can be easily identified when processing the image.

As a preferred example of the marker, the marking pen 200 can be applied. The component of the marking pen is a dye. The dye for marking according to the present invention comprises components that are harmless to human body and are easy to decompose. As a preferred example of the dye, a food dye can be applied.

The component of the marking pen includes a fluorescent material. The fluorescent material refers a material that emits visible rays or infrared rays by absorbing electromagnetic waves or ultraviolet rays.

The fluorescent material emitting infrared rays is easy to use in image processing, because it is easily distinguished by using an image sensor having an infrared filter. Therefore, when using the fluorescent material emitting infrared rays and the image sensor having an infrared filter, it becomes easier and more precise to distinguish the dye than to distinguish in visible rays.

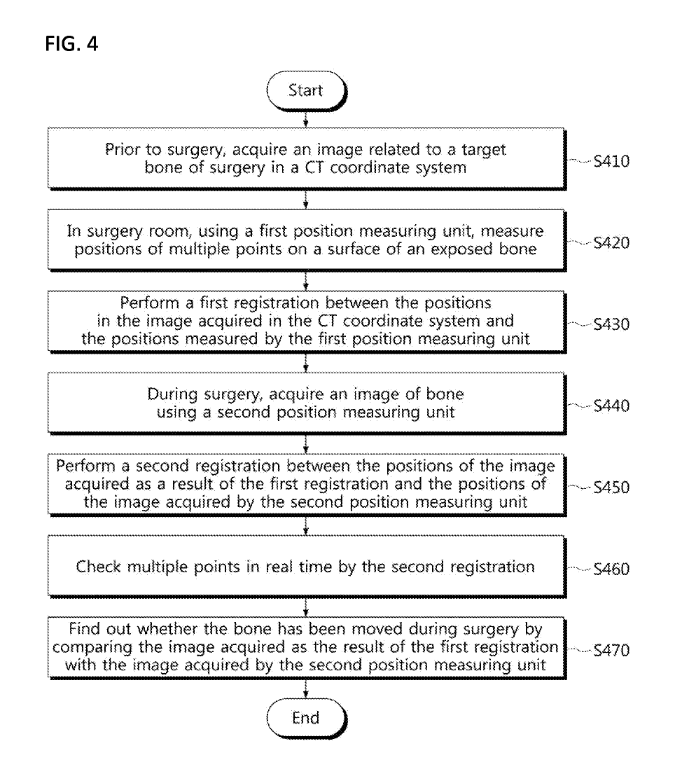

FIG. 4 is a flowchart that depicts the registration method of tissue position according to the present invention.

In explanation about an embodiment hereinafter, although it can be understood that each step of the method for registering positions of tissues according to the present invention is performed in corresponding elements of the apparatus for registering positions of tissues, which has been explained through FIG. 1, the each step of the method should be limited as function itself, which defines the each step. In other words, the performer of each step is not limited by the names of elements that are given as examples of performer of each step.

According to the method for registering positions of tissues, in step S410, prior to surgery, acquire an image related to a target bone of surgery in a CT coordinate system. Then, in step S420, when the surgery begins and the lesion region is opened, in surgery room, measure positions of multiple points on the surface of exposed bone using the first position measuring unit. The multiple points are dyed with a dye to be distinguished from other points on the bone. The dye could include at least one of a fluorescent material or a food dye, and the fluorescent material could include a fluorescent material emitting infrared ray.

In step S430, perform a first registration between the positions in the image acquired in the CT coordinate system and the positions measured by the first position measuring unit. In step S440, during surgery, acquire an image of bone by using the second position measuring unit. Then, in step S450, perform a second registration between the positions of the image acquired as the result of the first registration and the positions of the image acquired by the second position measuring unit.

In step S460, check the multiple points in real time through the second registration. Then, in step S470, find out whether the bone has been moved during surgery by comparing the image acquired as the result of the first registration with the image acquired by the second position measuring unit.

According to the present invention that has been described above with the embodiments, it is possible to establish a reference point for position registration without damaging to a bone in robotic surgery.

In addition, in compared with conventional physical methods, the time for establishing the reference points of registration is reduced, thereby enabling an efficient position registration.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. An apparatus for registering positions of tissues, comprising:
an image storage unit storing an examined image associated with a target bone of surgery;
a first position measuring unit measuring position values of multiple points on a surface of an exposed bone;
a second position measuring unit acquiring an image of the exposed bone; and
a registration control unit for performing a first registration between the positions obtained from the examined image associated with the target bone of surgery and the positions measured by the first position measuring unit, and for performing a second registration between the positions of the image acquired as a result of the first registration and the positions of the image acquired by the second position measuring unit;
wherein the multiple points are dyed with a dye to be distinguished from other points on the bone.

2. The apparatus according to claim 1, wherein the dye includes at least one of a fluorescent material or a food dye.

3. The apparatus according to claim 2, wherein the fluorescent material includes a fluorescent material emitting infrared rays.

4. The apparatus according to claim 1,
wherein the registration control unit checks the multiple points in real time by the second registration, and finds out whether the bone has been moved during surgery by comparing the image acquired as the result of the first registration with the image acquired by the second position measuring unit.

5. The apparatus according to claim 1, wherein the first position measuring unit includes a digitizer that has a tip contacted with the multiple points.

6. The apparatus according to claim 3, wherein the second position measuring unit includes an image sensor having an infrared filter.

7. A method for registering positions of tissues, comprising:
measuring positions of multiple points on a surface of an exposed bone by a first position measuring unit;
performing a first registration between positions obtained from an examined image associated with a target bone of surgery and the positions measured by the first position measuring unit;
acquiring an image of the exposed bone by a second position measuring unit;
performing a second registration between the positions of an image acquired as a result of the first registration and the positions of the image acquired by the second position measuring unit; and
wherein the multiple points are dyed with a dye to be distinguished from other points on the bone.

8. The method according to claim 7, wherein the dye includes at least one of a fluorescent material or a food dye.

9. The method according to claim 8, wherein the fluorescent material includes a fluorescent material emitting infrared rays.

10. The method according to claim 7, wherein the first position measuring unit includes a digitizer that has a tip contacted with the multiple points.

11. The method according to claim 7, wherein the second position measuring unit includes an infrared sensing camera.

12. The method according to claim 7, further comprising:
checking the multiple points in real time by the second registration.

13. The method according to claim 12, further comprising:
finding out whether the bone has been moved during surgery by comparing the image acquired as the result of the first registration with the image acquired by the second position measuring unit.
